(19) United States
(12) Patent Application Publication
(10) Pub. No.: US 2025/0332345 A1
(43) Pub. Date: Oct. 30, 2025

(54) Barrier Coated Sequential Stopper for a Multi-Chamber Syringe

(71) Applicant: Becton, Dickinson and Company, Franklin Lakes, NJ (US)

(72) Inventor: Alfred W. Prais, Hewitt, NJ (US)

(21) Appl. No.: 18/867,829

(22) PCT Filed: Oct. 23, 2023
(86) PCT No.: PCT/US2023/077519

§ 371 Date: Nov. 21, 2024

Related U.S. Application Data
(60) Provisional application No. 17/972,620, filed on Oct. 25, 2022.

(51) Int. Cl. A61M 5/19; A61M 5/178

(52) U.S. Cl. CPC A61M 5/19; A61M 2005/1787; A61M 2205/0238

(57) ABSTRACT

A stopper for a multi-chamber syringe includes a body having a first end surface, a second end surface, a peripheral surface extending between the first end surface and the second end surface, and at least one slit extending through the body for permitting fluid flow through the body of the stopper. The stopper also includes a first barrier film adhered to the first end surface of the body and a second barrier film adhered to the second end surface of the body. The first barrier film and/or the second barrier film comprise slits or openings aligned with the slit extending through the body for permitting fluid flow through the stopper. A multi-chamber syringe including the stopper and a method of making the stopper are also provided herein.

### CROSS-REFERENCE TO RELATED APPLICATION

The present application claims priority to U.S. Utility application Ser. No. 17/972,620 entitled “Barrier Coated Sequential Stopper for a Multi-Chamber Syringe” filed Oct. 25, 2022, the entire disclosure of which is hereby incorporated by reference in its entirety.

### BACKGROUND OF THE INVENTION

### Field of the Invention

The present disclosure relates generally to a stopper configured to be used with syringes containing doses of multiple fluids and, in particular, to a stopper for a syringe that provides sequential expulsion of a first or initial fluid, such as a first type of a medical fluid, followed by a secondary fluid, such as another type of medical fluid or a flush solution.

### Description of Related Art

Multi-chamber syringes can be used for sequential delivery of a dose of an initial medical fluid followed by a dose of a secondary medical fluid. Conventional multi-chamber syringes generally include a syringe barrel with a proximal chamber and a distal chamber separated by a stopper, seal, valve, or film for separating the initial fluid from the secondary fluid. Conventional multi-chamber syringes also include a plunger mechanism for expelling the initial fluid and the secondary fluid from the syringe barrel in sequence. Examples of dual-chamber syringes comprising conventional syringe barrels and mechanisms for expelling the initial fluid followed by the secondary fluid from the syringe barrel are described in U.S. Pat. No. 9,940,114, entitled “Dual-chamber syringe and associated connecting systems” and U.S. Pat. No. 9,833,572, entitled “Modular dual chamber syringe system.”

Multi-chamber syringes can include or can be connected to a needle cannula, such as a pen needle with a sharp tip, for direct injection of the initial medical fluid followed by the secondary medical fluid to vasculature of a patient. Alternatively, multi-chamber syringes can be configured to be connected to a vascular access device (VAD), such as intravenous (IV) catheter (e.g., a peripheral catheter or central venous catheter), or another fluid delivery device, for sequential delivery of the initial medical fluid followed by the secondary medical fluid through the VAD.

In some cases, multi-chamber syringes are used for delivery of a first type of therapeutic agent or drug followed by a second type of therapeutic agent or drug. Multi-chamber syringes can also be used for providing a flush solution through the VAD before and/or after the therapeutic agent. In particular, flush procedures can be performed prior to or following injection of the therapeutic agent to confirm catheter patency, avoid drug incompatibilities, ensure that the complete drug dose is administered to the patient, prevent thrombus formation, and/or to minimize a risk of bloodstream infections caused by contamination of the VAD. Further, flushing can prevent build-up of deposits of blood, blood residue, and IV drugs within a catheter or other VAD device.

Multi-chamber syringes can be provided as prefilled syringes where the chambers of the syringe are filled with the initial fluid and/or the secondary fluid during manufacture and prior to shipping to a medical facility for use. In such cases, it can be important that fluid contacting surfaces of the multi-chamber syringe are sealed and/or coated with biocompatible protective coatings or layers to prevent deterioration or contamination of the medical fluid(s) contained in the syringe prior art to use.

A problem with conventional multi-chamber syringes is that it can be difficult to coat all surfaces of the syringe, stopper, and other components that may contact fluids in the prefilled syringe to avoid contamination. The stoppers, syringes, and assembly methods of the present disclosure are provided to address these issues.

### SUMMARY OF THE INVENTION

According to an aspect of the disclosure, a stopper for a multi-chamber syringe includes a body having a first end surface, a second end surface, a peripheral surface extending between the first end surface and the second end surface, and at least one slit extending through the body for permitting fluid flow through the body of the stopper. The stopper also includes a first barrier film adhered to the first end surface of the body and a second barrier film adhered to the second end surface of the body. The first barrier film and/or the second barrier film comprise slits or openings aligned with the slit extending through the body for permitting fluid flow through the stopper.

According to another aspect of the disclosure, a multi-chamber syringe for sequential expulsion of at least an initial fluid followed by a secondary fluid includes a barrel having a first end, a second end comprising a fluid port for expulsion of the initial fluid and the secondary fluid from the barrel, and a sidewall extending between the first end and the second end of the barrel. The syringe also includes a first stopper and a second stopper slidably positioned in the barrel. The second stopper can include features of any of the previously described stoppers for multi-chamber syringes. The first stopper and the second stopper define a first chamber of the syringe barrel between the first stopper and the second stopper configured to contain the secondary fluid and a second chamber between the second stopper and the second end of the syringe barrel configured to contain the initial fluid.

According to another example of the disclosure, a prefilled syringe includes the previously described multi-chamber syringe and a predetermined volume of the secondary fluid disposed in the first chamber of the prefilled syringe. The prefilled syringe is provided with the first chamber fluidly isolated from the second chamber, thereby containing the predetermined volume of the secondary fluid within the prefilled syringe.

According to another aspect of the disclosure, a method for sequential expulsion of fluids from the previously described prefilled syringe includes a step of moving a plunger rod fixedly connected to the first stopper of the syringe in a proximal direction, which moves the second stopper of the syringe in the proximal direction, to aspirate the initial fluid into the second chamber of the syringe barrel. The method also includes once a dose of the initial fluid is in the second chamber, moving the plunger rod in the distal direction, as a single continuous stroke, thereby causing the initial fluid followed by the secondary fluid to be expelled from the fluid port of the syringe barrel.

According to another example of the present disclosure, a molding method for the previously described stopper for a multi-chamber syringe includes a step of molding a first part and a second part of the body of the stopper to a partially molded state. The method also includes steps of connecting a first barrier film to a first end of the partially-molded first part and connecting a second barrier film to a second end of the partially-molded second part. The method also includes a step of adhering the first part to the second part in a finishing mold, thereby forming the stopper comprising the body with the first and second barrier films laminated to the body. Finally, the method includes a step of forming the slit through the body and through the first barrier film and/or the second barrier film.

Non-limiting illustrative examples of embodiments of the present disclosure will now be described in the following numbered clauses.

Clause 1: A stopper for a multi-chamber syringe, the stopper comprising: a body comprising a first end surface, a second end surface, a peripheral surface extending between the first end surface and the second end surface, and at least one slit extending through the body for permitting fluid flow through the body of the stopper; a first barrier film adhered to the first end surface of the body; and a second barrier film adhered to the second end surface of the body, wherein the first barrier film and/or the second barrier film comprise slits or openings aligned with the slit extending through the body for permitting fluid flow through the stopper.

Clause 2: The stopper of clause 1, further comprising at least one protrusion extending axially from the proximal end of the body for separating the stopper from another stopper of a multi-chamber syringe.

Clause 3: The stopper of clause 1 or clause 2, wherein the peripheral surface of the body comprises at least one rib configured to contact an inner surface of a sidewall of a syringe barrel and at least one recessed portion configured to be spaced apart from the inner surface of the sidewall of the syringe barrel.

Clause 4: The stopper of any of clauses 1-3, wherein the first stopper and/or the second stopper comprise a thermoplastic elastomer, such as at least one of silicone, polypropylene, polyethylene, or synthetic or natural rubber (e.g., isoprene).

Clause 5: The stopper of any of clauses 1-4, wherein the body further comprises a cavity extending axially through a portion of the body from the first end toward the second end of the body.

Clause 6: The stopper of clause 5, wherein the cavity comprises an open first end, a closed second end with a substantially flat second surface, and an annular side surface between the first end and the second end, wherein the annular side surface comprises concave and convex portions.

Clause 7: The stopper of clause 6, wherein the slit extending through the body extends between the flat second surface of the cavity and the second end surface of the body.

Clause 8: The stopper of any of clauses 1-7, wherein the at least one slit extends substantially transverse to a central longitudinal axis of the body.

Clause 9: The stopper of any of clauses 1-8, wherein the body is made from two separately molded parts mated together by a subsequent molding process.

Clause 10: The stopper of clause 9, wherein the two separately molded parts of the body are each formed by a one-shot injection molding process.

Clause 11: The stopper of any of clauses 1-10, wherein the stopper is configured to transition between (i) a closed position, where fluid flow through the slit of the body is prevented, when a fluid pressure in the syringe barrel is below a predetermined opening pressure, and (ii) an open position when the fluid pressure in the syringe barrel is greater than or equal to the predetermined opening pressure, thereby establishing fluid communication between chambers of a syringe barrel through the stopper.

Clause 12: The stopper of any of clauses 1-11, wherein the slit of the body is biased to and initially provided in the closed position.

Clause 13: The stopper of any of clauses 1-12, wherein the first barrier film and/or the second barrier film comprise disks with a diameter matching a diameter of the first end surface or the second end surface of the body.

Clause 14: The stopper of any of clauses 1-13, wherein the first barrier film extends over an annular surface of the body and surfaces of a cavity defined by the body.

Clause 15: The stopper of any of clauses 1-14, wherein the second barrier film extends over a flat or substantially surface of the body.

Clause 16: The stopper of any of clauses 1-15, wherein the first barrier film and/or the second barrier film comprise a fluoropolymer, such as polytetrafluoroethylene (PTFE).

Clause 17: The stopper of any of clauses 1-16, wherein the peripheral surface of the body is free from barrier films.

Clause 18: The stopper of any of clauses 1-17, wherein the peripheral surface of the body is uncoated.

Clause 19: The stopper of any of clauses 1-18, wherein the first barrier film and/or the second barrier film have a thickness of from about 0.013 mm to about 0.25 mm.

Clause 20: A multi-chamber syringe for sequential expulsion of at least an initial fluid followed by a secondary fluid, the syringe comprising: a barrel comprising a first end, a second end comprising a fluid port for expulsion of the initial fluid and the secondary fluid from the barrel, and a sidewall extending between the first end and the second end of the barrel; a first stopper slidably positioned in the barrel; and a second stopper comprising the stopper of any of clauses 1-19 slidably positioned in the barrel, with the first stopper and the second stopper define a first chamber of the syringe barrel between the first stopper and the second stopper configured to contain the secondary fluid and a second chamber of the syringe barrel between the second stopper and the second end of the syringe barrel configured to contain the initial fluid.

Clause 21: The syringe of clause 20, wherein the slit of the body is configured to transition between (i) a closed position, where fluid flow through the slit of the body is prevented, when a fluid pressure in the first chamber is below a predetermined opening pressure, and (ii) an open position when fluid pressure in the first chamber is greater than or equal to the predetermined opening pressure, thereby establishing fluid communication between the first chamber and the second chamber through the slit of the second stopper.

Clause 22: The syringe of clause 20 or clause 21, wherein movement of the first stopper through the barrel in a proximal direction causes the second stopper to move in the proximal direction to aspirate the initial fluid into the second chamber through the fluid port of the barrel.

Clause 23: The syringe of any of clauses 20-22, wherein advancing the first stopper through the barrel causes the second stopper to move through the barrel to expel the initial fluid from the second chamber through the fluid port of the barrel.

Clause 24: The syringe of any of clauses 20-23, wherein, with the second stopper in a distal-most position, movement of the first stopper towards the second stopper causes fluid pressure in the first chamber to increase towards a predetermined opening pressure for the slit of the body.

Clause 25: The syringe of clause 24, wherein, once the slit opens, continued distal movement of the first stopper towards the second stopper moves the secondary fluid from the first chamber, through the slit of the body of the second stopper, the second chamber, and the fluid port of the barrel, thereby expelling the second fluid from the barrel.

Clause 26: The syringe of any of clauses 20-25, wherein the barrel comprises at least one of polyester, polycarbonate, polypropylene, polyethylene, polyethylene terephthalate, or acrylonitrile butadiene styrene.

Clause 27: The syringe of any of clauses 20-26, wherein the first stopper and/or the second stopper comprise a thermoplastic elastomer, such as at least one of silicone, polypropylene, polyethylene, or synthetic or natural rubber (e.g., isoprene).

Clause 28: The syringe of any of clauses 20-27, wherein the slit of the body of the second stopper is biased to and initially provided in the closed position.

Clause 29: The syringe of any of clauses 20-28, further comprising a plunger rod connected to the first stopper for moving the first stopper through the barrel.

Clause 30: The syringe of clause 29, wherein the syringe is configured such that the initial fluid followed by the secondary fluid are expelled from the syringe barrel by a single continuous advancement of the plunger rod distally into the syringe barrel.

Clause 31: The syringe of clause 29 or clause 30, wherein the syringe is configured such that the initial fluid followed by the secondary fluid are expelled from the syringe barrel by single-handed operation of the plunger rod.

Clause 32: The syringe of any of clauses 29-31, wherein the plunger rod comprises a proximal end comprising a press plate, a distal end comprising a threaded connector, and a body extending between the proximal end and the distal end.

Clause 33: The syringe of clause 32, wherein the first stopper comprises a threaded cavity that engages the threaded connector of the plunger rod, thereby fixing the plunger rod to the second stopper.

Clause 34: The syringe of any of clauses 29-33, wherein the stoppers are configured such that moving the plunger rod in the proximal direction or the distal direction moves both the first stopper and the second stopper.

Clause 35: The syringe of any of clauses 29-34, wherein the plunger rod is fixedly connected to the first stopper and the plunger rod and/or first stopper are free from connections to the second stopper.

Clause 36: The syringe of any of clauses 29-35, further comprising a removable plunger cap engaged between the plunger rod and the barrel for preventing movement of the plunger rod until the removable plunger cap is removed from the syringe.

Clause 37: A prefilled syringe, comprising: the syringe of any of clauses 20-36; and a predetermined volume of the secondary fluid disposed in the first chamber of the prefilled syringe, wherein the prefilled syringe is provided with the first chamber fluidly isolated from the second chamber, thereby containing the predetermined volume of the secondary fluid within the prefilled syringe.

Clause 38: The prefilled syringe of clause 37, wherein the secondary fluid comprises a saline and/or heparin flush solution.

Clause 39: The prefilled syringe of clause 37 or clause 38, wherein the syringe further comprises a plunger rod connected to the first stopper for moving the first stopper and/or the second stopper through the barrel.

Clause 40: The prefilled syringe of clause 39, further comprising a removable plunger cap engaged between the plunger rod and the barrel of the syringe for preventing movement of the plunger rod until the cap is removed, thereby maintaining fluid isolation of the first chamber and the second chamber until the plunger cap is removed.

Clause 41: A method for sequential expulsion of fluids from the prefilled syringe of any of clauses 37-40, the method comprising: moving a plunger rod fixedly connected to the first stopper of the syringe in a proximal direction, which moves the second stopper of the syringe in the proximal direction, to aspirate the initial fluid into the second chamber of the syringe barrel; and once a dose of the initial fluid is in the second chamber, moving the plunger rod in the distal direction, as a single continuous stroke, thereby causing the initial fluid followed by the secondary fluid to be expelled from the fluid port of the syringe barrel.

Clause 42: The method of clause 41, wherein the single continuous stroke of the plunger rod is performed as a single-hand operation.

Clause 43: The method of clause 41 or clause 42, wherein aspirating the initial fluid into the second chamber comprises attaching a needle to the fluid port of the syringe, inserting the needle into a fluid container, and drawing the initial fluid from the fluid container to the second chamber of the syringe.

Clause 44: The method of any of clauses 41-43, further comprising attaching at least one patient line to the fluid port of the syringe barrel prior to moving the plunger rod in the distal direction, wherein the fluid expelled from the syringe passes to the patient through at least one patient line.

Clause 45: The method of any of clauses 41-44, wherein the initial fluid comprises a therapeutic agent.

Clause 46: A molding method for the stopper of any of clauses 1-19, the method comprising: molding a first part and a second part of the body of the stopper to a partially molded state; connecting a first barrier film to a first end of the partially-molded first part; connecting a second barrier film to a second end of the partially-molded second part; adhering the first part to the second part in a finishing mold, thereby forming the stopper comprising the body with the first and second barrier films laminated to the body; and forming the slit through the body and through the first barrier film and/or the second barrier film.

Clause 47: The method of clause 46, wherein the first part and the second part are molded by a one-shot injection molding process.

Clause 48: The method of clause 46 or clause 47, wherein connecting the first barrier film to the first molded part comprises attaching a flat sheet of a barrier material to the first end of the first part and trimming the flat sheet to match a shape of the first end of the first part.

Clause 49: The method of clause 48, wherein connecting the second barrier film to the second end of the second part comprises attaching a flat sheet of a barrier material to the second end of the second part and trimming the flat sheet to match a shape of the second end of the second part.

Clause 50: The method of any of clauses 46-49, wherein adhering the parts together comprises curing the parts to a temperature of about 50° C. to about 70° C.

Clause 51: The method of any of clauses 46-50, wherein the first barrier film and/or the second barrier film comprise a fluoropolymer.

Clause 52: The method of any of clauses 46-51, wherein the first part and the second part are molded together along uncoated surfaces of the respective parts.

Clause 53: The method of any of clauses 46-52, wherein adhering the first part to the second part comprises placing the first part in a first half of the mold, placing the second part in a second half of the mold, and closing the mold to mate the parts together along uncoated surfaces of the parts.

Clause 54: The method of clause 53, wherein a part line between the parts is relieved by about 0.05 mm to about 0.2 mm.

Clause 55: The method of clause 53 or clause 54, wherein adhering the parts together comprises applying sufficient temperature and pressure to the mold to form a single integral body from the first part and the second part.

### BRIEF DESCRIPTION OF THE DRAWINGS

FIG. 1A is a perspective view of a multi-chamber syringe, according to an aspect of the present disclosure.

FIG. 1B is a perspective exploded view of the multi-chamber syringe of FIG. 1A.

FIG. 2A is a perspective view of a stopper of the multi-chamber syringe of FIG. 1A, according to an aspect of the disclosure.

FIG. 2B is a top plan view of the stopper of FIG. 2A.

FIG. 2C is a cross-sectional view of the stopper of FIG. 2A.

FIG. 3A is a side view of the multi-chamber syringe of FIG. 1A in an initial or partially filled position.

FIG. 3B is a side view of the multi-chamber syringe of FIG. 1A in a filled positon.

FIG. 3C is a cross-sectional view of the multi-chamber syringe of FIG. 1A with a filled proximal chamber and an empty distal chamber.

FIG. 3D is a side view of the multi-chamber syringe of FIG. 1A in a final position after expulsion of the initial fluid and the secondary fluid from the syringe.

FIG. 4 is a flow chart showing a method for expelling medical fluids from the multi-chamber syringe of FIG. 1A.

FIG. 5 is a flow chart showing a method for forming a stopper with barrier coatings, according to an aspect of the disclosure.

FIGS. 6A-6E are schematic drawings showing steps of a method for making a stopper with barrier films, which can be used in a multi-chamber syringe, according to aspects of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”. “vertical”, “horizontal”, “top”, “bottom”, “lateral”, “longitudinal”, and derivatives thereof shall relate to the invention as it is oriented in the drawing figures. As used herein, the term “proximal” refers to a portion or end of a device, such as a syringe or catheter, which is grasped, manipulated, or used by a practitioner or another user. The term “distal” refers to an end or portion of the device that is farthest away from the portion of the device that is grasped, manipulated, or used by the practitioner. For example, the “proximal end” of a syringe refers to the portion of the syringe that the practitioner pushes on to advance a stopper through a syringe barrel. The “distal end” of the syringe refers to the end that is attached to a vascular access device or which includes a needle cannula configured to be inserted through a patient's skin. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to pistons, plungers, or stoppers 110, 112 for use with a multi-chamber syringe 10. One of the stoppers, referred to herein as a distal stopper 112, can be configured to divide a drug container, such as a syringe barrel, into a distal chamber, which contains a first or initial fluid to be injected to a patient, and a proximal chamber, which contains a subsequent or secondary fluid to be delivered to the patient after the initial fluid. In some examples, the distal stopper 112 comprises a built-in check valve that allows the secondary fluid contained in the proximal chamber of the syringe 10 to pass through the distal stopper 112 only after the initial fluid is expelled from a distal chamber of the syringe 10. Accordingly, the multi-chamber syringe 10 of the present disclosure can be used for fluid delivery of multiple medical fluids to vasculature of a patient in sequence through a needle cannula inserted into a blood vessel of the patient with only one needle stick and no additional fluid delivery steps to be performed by the practitioner. In other examples, the multi-chamber syringe 10 of the present disclosure can allow for delivery of multiple medical fluids to a patient in sequence through a VAD without needing to attach multiple syringes or fluid containers to the VAD.

As described in further detail herein, the distal stopper 112 can also include a leachable/extractable barrier layer, coating, or film over fluid contacting surfaces of the distal stopper 112. Specifically, a first or proximal barrier coating or film can be positioned on the distal stopper 112 to contact fluid contained in the proximal chamber and a second or distal barrier coating or film can be positioned to contact fluid contained in the distal chamber of the syringe 10. Including a barrier layer, coating, or film over both fluid contacting surfaces of the distal stopper 112 means that the syringe 10 can be used with sensitive medical fluids, such as pharmaceuticals that are likely to degrade when placed in prolonged contact other plastic materials.

The present disclosure is also directed to features of a prefilled or partially prefilled multi-chamber syringe 10 including the proximal stopper 110 and the distal stopper 112. In particular, the multi-chamber syringe 10 can be configured to expel the initial fluid followed by the secondary fluid from a syringe barrel through a fluid port or nozzle of the syringe 10. As previously described, the initial fluid can be a medical fluid, which, as used herein, can refer to a medication, total parenteral nutrient (TPN) liquid, or another therapeutic agent used for treatment of chronic or acute conditions, as are known in the art. Exemplary therapeutic agents can include, for example, drugs, chemicals, biological, or biochemical substances that, when delivered in a therapeutically effective amount to the patient, achieve a desired therapeutic effect. The secondary fluid can be another medical fluid, such as another type of therapeutic agent or drug. The secondary fluid can also be a flush solution, such as saline and/or a heparin lock flush solution. An example of a saline flush solution is 0.9% sodium chloride USP for injection. An example of a heparin lock flush solution is 0.9% sodium chloride with 100 USP units of heparin sodium per mL or 10 USP units of heparin sodium per mL. Other flush solutions, as are known in the art, may also be used with the syringes 10 of the present disclosure.

The syringes 10 of the present disclosure allow a practitioner, such as a medical technician, nurse, physician assistant, physician, or other trained or untrained clinicians or medical caregivers, to administer the initial fluid followed by the secondary fluid without needing to change syringes or fluid containers between delivery of the initial fluid and the secondary fluid. Further, the syringes 10 of the present disclosure allow the practitioner to provide the sequential delivery of the initial fluid followed by the secondary fluid through a single continuous advancement of a plunger rod of the syringes 10.

As used herein, “single continuous advancement of a plunger rod” means that the practitioner is able to push the plunger rod in a distal direction, through a barrel of the syringe 10, as a single continuous stroke to expel the initial fluid followed by the secondary fluid from the syringe barrel. The practitioner does not need, for example, to perform multiple needle sticks or to disconnect a syringe or another device from the VAD between delivery of the initial fluid and the secondary fluid. Further, using the syringes 10 of the present disclosure, the practitioner does not need to perform any other action, such as twisting, rotating, or pulling on the plunger rod or pressing another component or mechanism of the syringe 10, in order to perform the sequential delivery of the initial fluid and the secondary fluid. Accordingly, the fluids can be expelled from the syringe 10 in sequence in response solely to the single continuous stroke of the plunger rod in the distal direction by the practitioner, which can be performed as a “single-handed” operation or movement (i.e., the practitioner can hold the syringe 10 and press the plunger rod through the barrel with one hand). Accordingly, the syringes 10 of the present disclosure simplify processes for administering the initial fluid followed by the secondary fluid to a VAD and/or patient compared to conventional fluid delivery practices.

In some examples, the syringes 10 are provided as a partially prefilled syringe, where a chamber of the syringe 10 is filled with a flush solution or another medical fluid during manufacturing. The partially prefilled syringe 10 can include caps, clips, retainers, and/or other packaging to hold the plunger rod in place and to ensure that the flush solution does not leak from the partially prefilled syringe 10 at unexpected times, such as during shipping.

In some examples, the syringes 10 of the present disclosure can be configured to allow the practitioner to aspirate a medical fluid into the syringe 10 prior to fluid delivery to the patient. For example, the practitioner can insert a nozzle or needle of the syringe 10 into a vial containing the initial fluid (e.g., the medical fluid) and then aspirate the medical fluid into a chamber of the syringe 10 by moving the plunger rod of the syringe 10 in a proximal direction. After the initial fluid is aspirated into the chamber of the syringe 10, sequential delivery of the initial fluid followed by the secondary fluid can occur by connecting a pen needle cannula to the syringe 10 or by connecting the syringe 10 to a fluid port of a VAD and then moving the plunger rod of the syringe 10 in the distal direction, thereby expelling the initial fluid followed by the secondary fluid from the syringe 10 to the VAD.

By eliminating the need to use different syringes for delivery of the initial fluid and the secondary fluid, the multi-chamber syringes 10 of the present disclosure simplify the fluid administration procedure, providing substantial time savings compared to conventional fluid administration practices. The syringes 10 of the present disclosure can also reduce infection risk and allow for flushing of the VAD immediately following administration of the medical fluid, which may prevent drug occlusion in the VAD.

The present disclosure is also directed to a molding process for making the distal stopper 112 including the barrier coatings or films. As described in further detail herein, the molding process can comprise forming separate parts by a one-shot injection molding process. The injection molded parts are then formed together in a finishing mold, which produces a stopper comprising a single integral body with the barrier coatings or films adhered to both proximal and distal surfaces of the body.

### Multi-Chamber Syringe for Sequential Drug Delivery

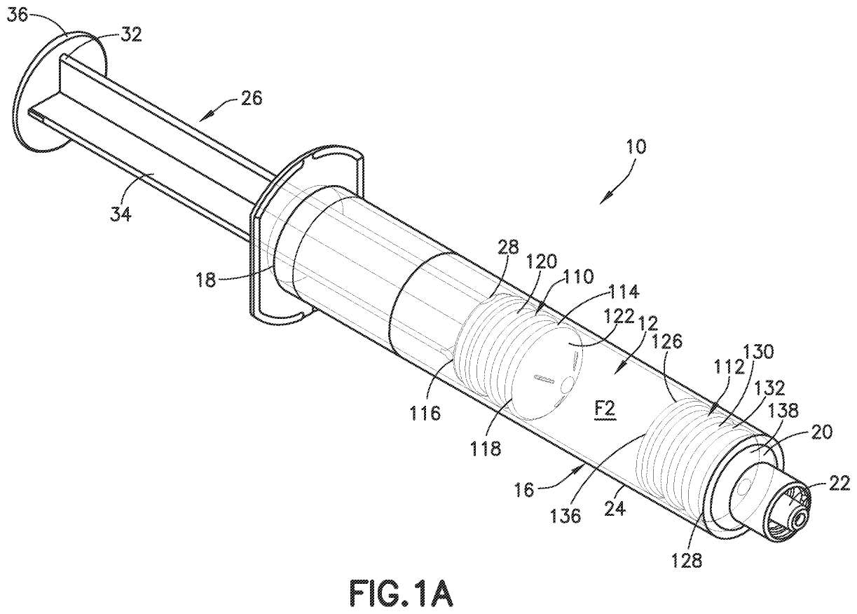

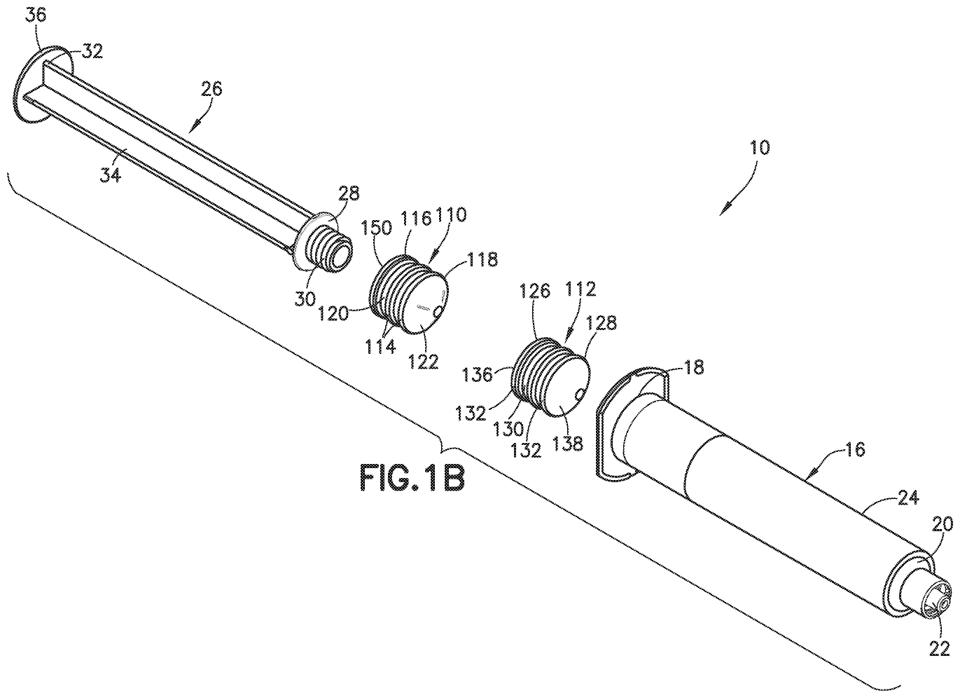

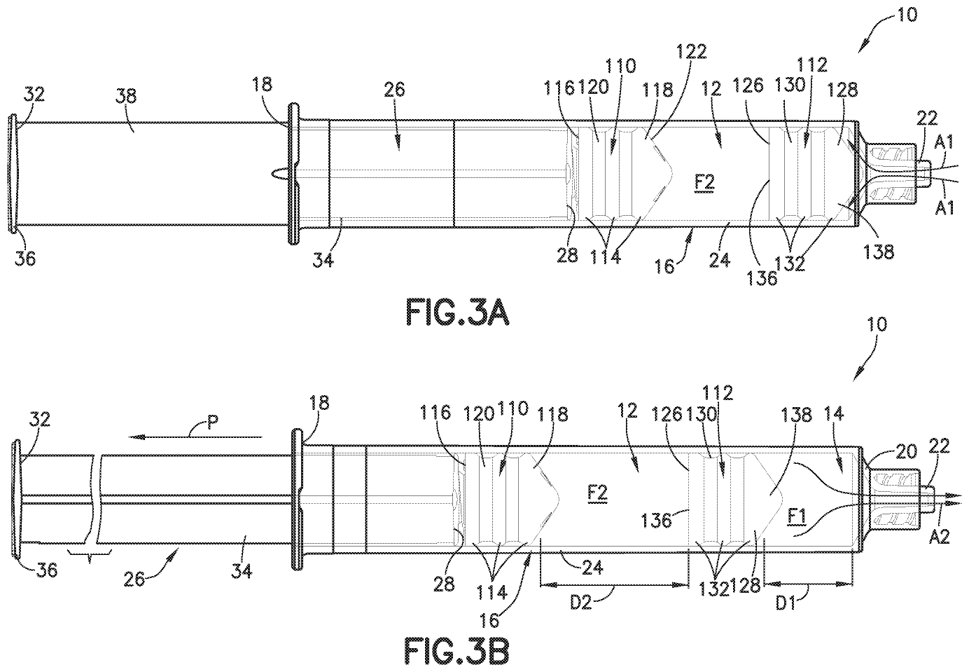

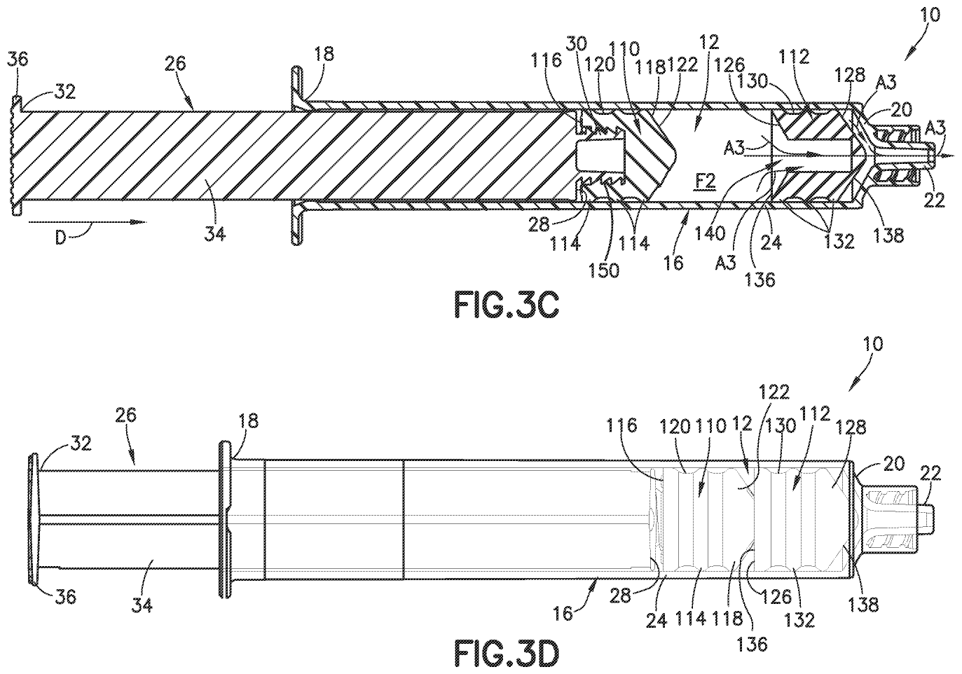

FIGS. 1A and 1B illustrate an example of a multi-chamber syringe 10 for sequential expulsion of at least an initial fluid F1 (shown in FIG. 3B) contained in the second or distal fluid chamber 14 followed by a secondary fluid F2 (shown in FIGS. 1A and 3A-3C) contained in a first or proximal fluid chamber 12. As previously described, the initial fluid F1 can be a medical fluid, such as a drug or another therapeutic agent intended for delivery to a patient through a needle cannula or through a VAD, such as a catheter or IV line. The secondary fluid F2 can be another type of therapeutic agent or a flush solution, such as saline solution and/or an anticoagulant, such as heparin. The type and amount of solution contained in the proximal chamber 12 and/or the distal chamber 14 may vary depending, for example, on the specific type of needle cannula, catheter, or IV line being used for an injection and/or on the therapeutic effect to be achieved. In some examples, the syringe 10 contains or is configured to contain between about 1 mL and 20 mL of the initial fluid F1 and/or the secondary fluid F2 or, preferably, between about 5 mL and about 10 ml of the initial fluid F1 and/or the secondary fluid F2.

In some examples, the syringe 10 comprises a barrel 16 having an open proximal end 18, a distal end 20 including a nozzle or fluid port 22 for expulsion of the initial fluid F1 and the secondary fluid F2 from the barrel 16, and a sidewall 24 extending between the proximal end 18 and the distal end 20 of the barrel 16. The fluid port 22 of the barrel 16 can be a connector, such as a luer connector, threaded connector, or snap connector, configured to be connected to a needle cannula for accessing, for example, an interior of a medical vial containing a medical fluid. The fluid port 22 of the syringe 10 can also be a needleless connector configured to be connected directly or indirectly to a fluid port, valve, or another terminal access portion of a VAD. For example, a common type of fluid port of a VAD is a pierceable septum or pre-slit septum made of rubber or another elastomeric material, which permits insertion of a sharp or blunt needle cannula in order to infuse fluids or to withdraw fluids from a catheter of the VAD. Another common fluid port of a VAD is a valve, which does not require a needle for accessing the VAD. Instead, the valve can be activated by a frusto-conically shaped tip of the syringe barrel 16 to provide fluid communication between the interior of the barrel 16 and the VAD.

In some examples, the barrel 16 of the syringe 10 can be substantially similar in shape, size, and configuration to barrels of syringes used for administering a flush solution to a VAD, as are known in the art. For example, the barrel 16 can be a cylindrical structure formed from a rigid thermoplastic material, such as polyester, polycarbonate, polypropylene, polyethylene, polyethylene terephthalate, acrylonitrile butadiene styrene, or other injection moldable or formable resin materials, as are known in the art. Exemplary barrels for syringes are described, for example, in U.S. Patent Appl. Pub. No. 2020/0061297, entitled “Flush Syringe Assembly with Controlled Pulsatile Flushing,” which is incorporated herein by reference in its entirety.

The syringe 10 further comprises the first or proximal stopper 110 and the second or distal stopper 112 slidably positioned within the barrel 16 of the syringe 10. The stoppers 110. 112 separate the barrel 16 into the proximal chamber 12 and the distal chamber 14. Specifically, as shown most clearly in FIG. 1A, the proximal chamber 12 is between a distal end of the proximal stopper 110 and a proximal end of the distal stopper 112. The distal chamber 14 (shown in FIG. 3C) is between a distal end of the distal stopper 112 and the distal end 20 of the barrel 16.

As described in further detail herein, the stoppers 110, 112 include many features of conventional syringe stoppers or plungers, as are known in the art. For example, the stoppers 110, 112 can be substantially cylindrical bodies formed from a flexible and/or deformable material, such as a thermoplastic elastomer material. Examples of thermoplastic elastomers include, but are not limited to, silicone, polypropylene, polyethylene, or synthetic or natural rubber (e.g., isoprene), or combinations thereof. The stoppers 110, 112 can include radially extending ribs 114, 132 or rings that seal against an inner surface of the syringe barrel 16 so that the stoppers 110, 112 can move fluids through the syringe barrel 16 towards the distal end 20 of the barrel 16.

More specifically, as shown in FIGS. 1A and 1B, the proximal stopper 110 can include a proximal end surface 116, a distal end surface 118, and an outer peripheral surface 120 extending between the proximal surface 116 and the distal surface 118. The proximal stopper 110 can further include one or more of the annular ribs 114 protruding from the outer peripheral surface 120. The annular ribs 114 are configured to seal against an inner surface of the sidewall 24 of the syringe barrel 16, ensuring that fluid moves through the syringe barrel 16 in an expected manner. In some examples, the proximal stopper 110 includes multiple annular ribs 114 in order to improve stability and to prevent the proximal stopper 110 from tilting, shifting, or otherwise deforming as the stopper 110 moves through the syringe barrel 16. For examples, as shown in FIGS. 1A and 1B, the proximal stopper 110 includes three ribs 114. In other examples, the proximal stopper 110 can include fewer than three ribs 114 or more than three ribs 114.

As shown in FIG. 1A, the distal end surface 118 of the proximal stopper 110 is a fluid contacting surface in contact with the second fluid F2 contained in the proximal chamber 12 of the syringe barrel 16. The proximal surface end 116 of the first stopper 110 does not contact fluid during normal and/or expected use of the syringe 10. In order to protect the fluid contained in the proximal chamber 12, the first stopper 110 can include a barrier coating, layer, or film covering, referred to herein as a distal barrier film 122, over the fluid contacting portion of the distal end surface 118 of the proximal stopper 110. An exemplary stopper including a barrier layer, which can be used as the proximal stopper 110 for the multi-chamber syringe 10 disclosed herein, is described in detail in PCT Appl. Pub. No. WO 2022/109214A1 (hereinafter “the '214 publication”), entitled “Barrier coated stopper and method of forming same,” which is incorporated herein by reference in its entirety.

As described in the '214 publication, the barrier film 122 can optionally be formed from a fluoropolymer material, such as a polymer formed from a polytetrafluoroethylene resin (PTFE), an ethylenetetrafluoroethylene resin (ETFE), expanded polytetrafluoroethylene (ePTFE), or from similar fluropolymer materials. Beneficially, expanded fluoropolymer structures can be sufficiently strong to form thin barriers, which remain intact during the forming process and following installation of the stopper 110 into the syringe barrel 16. Desirably, the fluoropolymer materials also should not exhibit detrimental effects to the therapeutic agent or to other medical solutions in the syringe barrel 16 and should be free of silicone-based materials, such as silicone oil. Further, the barrier film 122 material should be biocompatible and should have good mechanical integrity. Also, the barrier material should be inert and should be easy to process.

The distal stopper 112 is positioned in the syringe barrel 16 and separates the proximal chamber 12 from the distal chamber 14. As described herein, the distal stopper 112 includes barrier layers, coatings, or films on both fluid contacting surfaces for separating medical fluid contained in the proximal chamber 12 from medical fluid contained in the distal chamber 14. In some examples, the distal stopper 112 forms a one-way check valve for selectively controlling fluid flow between the proximal chamber 12 and the distal chamber 14. As used herein, a one-way check valve (also referred to as a non-return valve, retention valve, or one-way valve) refers to a valve that allows a flow of fluid through the valve in only one direction. For example, the distal stopper 112 can be configured to permit fluid flow from the proximal chamber 12 to the distal chamber 14, while fluid flow from the distal chamber 14 to the proximal chamber 12 is prevented. The one-way check valve can be, for example, a ball valve, diaphragm valve, tilting disc valve, clapper valve, or duckbill valve, as are known in the art.

In some examples, the check valve of the distal stopper 112 is a single use valve. As used herein, a “single-use” valve refers to a valve that is initially provided in a closed position and which transitions to an open position one time. The single-use valve does not return to the closed position after being opened. For example, the check valve can be configured to transition from the closed position to the open position when a fluid pressure of the proximal chamber 12 is equal to or greater than an activation or opening pressure of the distal stopper 112. Once the distal stopper 112 opens, the distal stopper 112 does not return to the closed position. An advantage of a single-use valve is that the fluid pressure of the proximal chamber 12 does not need to remain above the activation or opening pressure for the distal stopper 112 for the entire time that the secondary fluid F2 is being pushed through the distal stopper 112 to the distal chamber 14. Instead, the practitioner need only press a plunger rod connected to the proximal stopper 110 with sufficient force to create a fluid pressure sufficient to open the distal stopper 112. Once the distal stopper 112 is open, the practitioner can push the plunger rod with less force to expel the secondary fluid F2 from the proximal chamber 12 to the distal chamber 114 through the distal stopper 112 because the distal stopper 112 does not return to the closed position even when the fluid pressure in the proximal chamber 12 drops below the activation or opening pressure of the distal stopper 112.

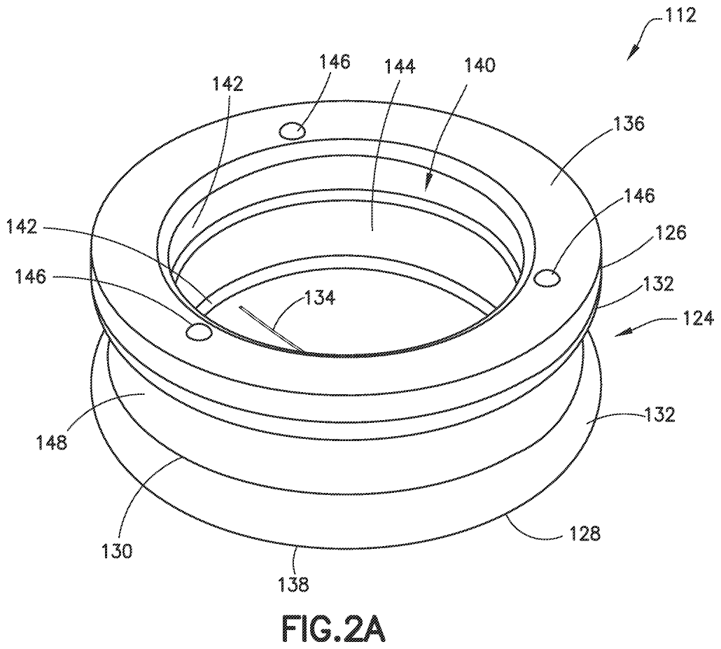

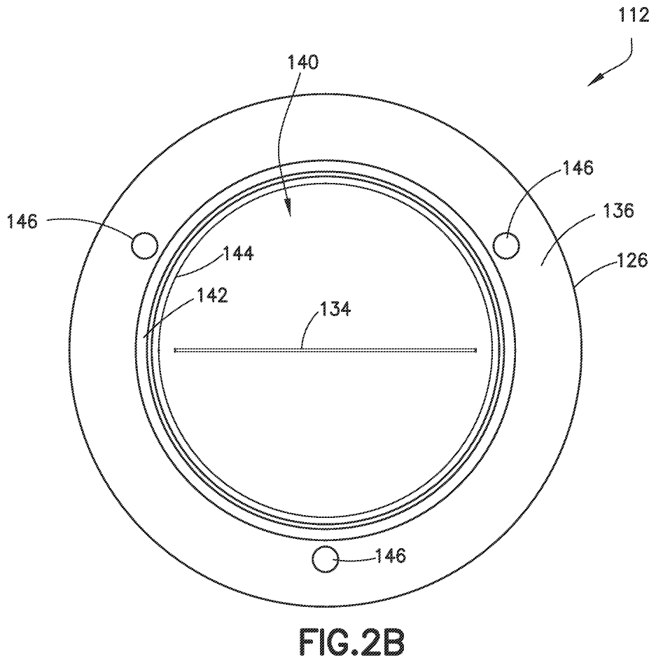

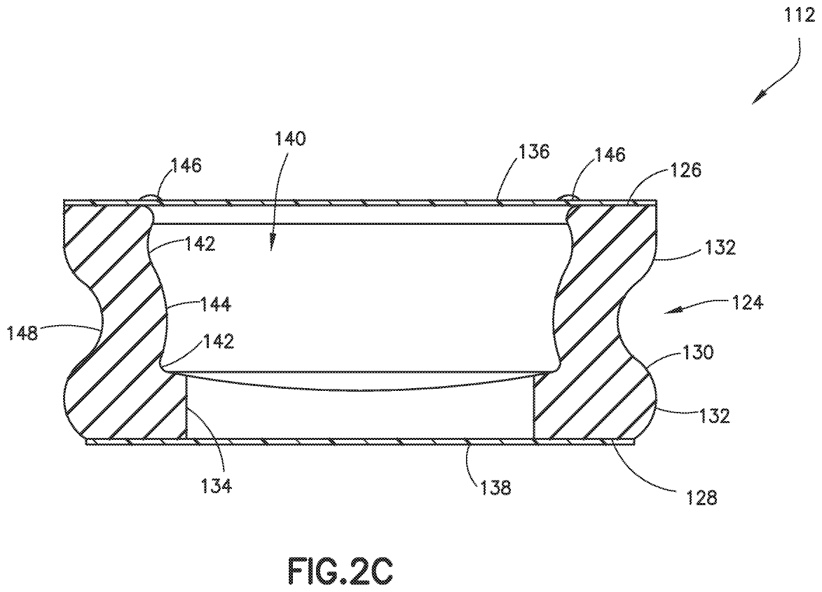

In some examples, as shown in FIGS. 1A and 1B, as well as in FIGS. 2A and 2B, the distal stopper 112 comprises a body 124 comprising a first or proximal end surface 126, a second or distal end surface 128, and a peripheral surface 130 extending between the proximal end surface 126 and the distal end surface 128. As described in further detail herein, the body 124 can be an integral structure formed from separately molded parts that are adhered, cured, or joined together during a final molding process. In some examples, the distal end surface 128 can be conical or frusto-conical to assist in expelling fluid from the syringe barrel 16 through the fluid port 22. Alternatively, the distal end surface 128 of the distal stopper 112 can be substantially flat, sloped, step-shaped, or other configurations depending, for example, on the size and shape of the syringe barrel 16. In some examples, the proximal end surface 126 of the distal stopper 112 can include a cavity 140, recessed area, or depression extending axially through a portion of the body 124 from the proximal end surface 126 toward the distal end surface 128. The cavity 140 can be similar in size and shape to cavities of conventional syringe stoppers configured to connect to plunger rods, as are known in the art. For example, as shown most clearly in FIGS. 2A and 2B, the cavity 140 can include an open proximal or first end, a closed second or distal end with a substantially flat distal surface, and an annular side surface between the proximal end and the distal end. The annular side surface of the cavity can include concave portions 142 and convex portions 144. In other examples, the annular side surface of the cavity 140 can be cylindrical, frusto-conical, or tapered.

As with the proximal stopper 110, the peripheral surface 130 of the distal stopper 112 can also include one or more radially extending ribs 132 configured to contact the inner surface of the syringe barrel 16, creating a seal between the distal stopper 112 and the inner surface of the syringe barrel 16. As previously described, multiple ribs 132 can be provided to improve stability of the distal stopper 112 as it advances or retracts through the syringe barrel 16 preventing the stopper 112 from tilting, shifting, or deforming during use. The peripheral surface 130 also includes one or more recessed portions 148 between the ribs 132 configured to be spaced apart from the inner surface of the sidewall 24 of the syringe barrel 16. For example, as shown in FIGS. 2A and 2B, the body 124 can include two ribs 132, with a recessed portion 148 between the two ribs 132. However, the number of ribs 132 may vary within the scope of the present disclosure. In some examples, the body 124 can include only one rib 132, while in other examples, the body 124 can include three or more ribs 132.

In some examples, the distal stopper 112 also includes one or more protrusions 146, such as detents or other raised bumps, extending axially from the proximal end surface 126 of the body 124. The protrusions 146 can be positioned on the proximal end surface 126 for creating a small gap between the distal surface 118 of the proximal stopper 110 and the proximal surface 130 of the distal stopper 112 when the proximal stopper 110 is pressed against the distal stopper 112, as shown, for example, in FIG. 3D.

In some examples, the body 124 also includes a slit 134 extending through the body 124 for permitting fluid flow through the body 124 when the distal stopper 112 and/or slit 134 is in an open position. The slit 134 can be an elongated, narrow opening extending between the distal surface of the cavity 140 and the distal end surface 128 of the body 124. In some examples, the body 124 includes a single slit 134 that extends across the distal end surface 128 substantially transverse to a central longitudinal axis of the body 124. In other examples, the body 124 can include multiple slits and/or can include slits 134 at other positions on the distal end surface 128 of the body 124.

The distal stopper 112 and/or slit 134 can be configured to transition between a closed position, where fluid flow through the distal stopper 112 and/or slit 134 of the body 124 is prevented, and an open position, where fluid flow through the distal stopper 112 and/or slit 134 can occur, thereby establishing fluid communication between the proximal chamber 12 and the distal chamber 14 through the distal stopper 112. In some examples, the distal stopper 112 can be configured to remain in the closed position when a fluid pressure in the proximal chamber 12 of the syringe barrel 16 is below a predetermined opening pressure. The distal stopper 112 can be configured to transition to the open position when the fluid pressure in the proximal chamber 12 of the syringe barrel 16 is greater than or equal to the predetermined opening pressure. In some examples, the slit 134 and/or the distal stopper 112 are biased to and initially provided in the closed position, meaning that when fluid pressure in the proximal chamber 12 is nominal or is substantially equal to fluid pressure in the distal chamber 14, the distal stopper 112 and/or the slit 134 are in the closed position. The distal stopper 112 and/or the slit 134 transition to the open position when the fluid pressure in the proximal chamber 12 substantially increases above the predetermined activation pressure value.

The activation or opening pressure for the distal stopper 112 or slit 134 can be selected based on fluid pressures that commonly occur when a stopper is manually moved through a barrel of a conventional syringe at a reasonable rate, as occurs when a practitioner pushes a plunger rod of a syringe through the syringe barrel. In some examples, the activation or opening pressure for the slit 134 and/or distal stopper 112 is at least 30 psi, though the activation or opening pressure can be selected or optimized for different syringe designs taking into account, for example, the size, shape, and materials of the stoppers 110, 112, the syringe barrel 16, and other components of the multi-chamber syringe 10. In some examples, the syringe 10 is configured such that the activation or opening pressure for the slit 134 and/or the distal stopper 112 is greater than a pressure required to infuse the drug (i.e., the pressure required to expel the initial fluid F1 from the distal chamber 14 through the fluid port 22 and to the patient through the needle cannula and/or through the VAD). Accordingly, the practitioner may be required to apply a greater force to a plunger rod of the syringe 10 connected to the proximal stopper 110 in order to cause the distal stopper 112 to open than is required to move the stoppers 110, 112 through the barrel 16. Due to these differences in the force that must be applied to the plunger rod, the practitioner can receive feedback (i.e., a feeling that increased force on the plunger rod is needed) indicating that the initial fluid F1 has been expelled from the syringe 10 and that the distal stopper 112 is in its distal-most position in the barrel 16.

The distal stopper 112 further comprises the films, coatings, layers, or other barriers between the fluid chambers 12, 14 and the proximal end surface 126 and the distal end surface 128 of the distal stopper 112. Specifically, the distal stopper 112 can comprise a first or proximal barrier film 136 adhered to the proximal end surface 126 of the body 124 and a second or distal barrier film 138 adhered to the distal end surface 128 of the body 124. In some examples, the proximal barrier film 136 also covers inner surfaces of the cavity 140 of the body 124. Alternatively, the proximal barrier film 136 can extend over or cover portions of an open end of the cavity 140, which extends through the proximal end surface 126 of the body 124. In some examples, the proximal barrier film 136 and/or the distal barrier film 138 can comprise slits or openings aligned with the slit 134 extending through the body 124 for permitting fluid flow through the distal stopper 124.

The barrier films 136, 138 can be formed from similar or the same materials as the distal barrier film 122 of the proximal stopper 110. For example, the barrier films 136, 138 of the distal stopper 112 can be formed from any of the barrier materials described in the '214 publication referred to previously, or from other biocompatible fluid impenetrable and/or hydrophobic materials, as are known in the art. In some examples, the barrier film 136, 138 can be formed from a fluoropolymer material, such as a polymer formed from a polytetrafluoroethylene resin (PTFE), an ethylenetetrafluoroethylene resin (ETFE), expanded polytetrafluoroethylene (ePTFE), or similar fluoropolymer materials.

In some examples, the barrier films 136, 138 are sized or trimmed to cover only the fluid contacting surfaces of the distal stopper 112. For example, the proximal barrier film 136 and the distal barrier film 138 can be trimmed into disk-shaped members having a diameter matching or substantially matching (e.g., within about 5% of) an outer diameter of the proximal end surface 126 and/or the distal end surface 128 of the distal stopper 112. The barrier films 136, 138 can be mounted or secured to the proximal end surface 126 and the distal end second surface 128 of the distal stopper 112 by various lamination and adhesion methods, as are known in the art. For example, a curable polymer material and/or a biocompatible adhesive can be used to secure the barrier films 136, 138 to the surfaces 126, 128 of the distal stopper 112. The peripheral surface 130 of the distal stopper 112 including the ribs 132 that contact the sidewall of the barrel 16, can be free from barrier layers, coatings, or films, so as not to disrupt the ability of the distal stopper 112 to seal against and/or slide against the sidewall 24 of the syringe barrel 16. In some examples, thickness of the barrier films 136, 138 can influence a fluid pressure required to cause the distal stopper 112 to transition to the open position. Accordingly, the thickness of the barrier films can be selected or modified to adjust the fluid pressure required to cause the stopper 112 to open or close. For example, the thickness of the proximal barrier film 136 and/or the distal barrier film 138 can be from about 0.013 mm to about 0.25 mm. In one configuration, a film that is melt-processible may be used so that it can be thermoformed simultaneously with the rubber vulcanization and/or molding step.

With continued reference to FIGS. 1A and 1B, as well as to FIGS. 3A-3D, the stoppers 110, 112 of the syringe 10 of the present disclosure are configured to move through the barrel 16 simultaneously and in a coordinated manner, even though the stoppers 110, 112 are not mechanically connected or engaged together. This simultaneous movement occurs due to the incompressible property of a liquid, such as the secondary fluid F2 (e.g., the second therapeutic agent or the flush solution), in the proximal chamber 12 of the barrel 16. More specifically, pressure applied to the proximal stopper 110 is transferred to a fluid column of the secondary fluid F2 in the proximal chamber 12. This fluid column applies pressure both to the distal stopper 112 causing the distal stopper 112 to move through syringe the barrel 16 toward the distal-most position (shown in FIGS. 3C and 3D), thereby expelling the initial fluid F1 from the distal chamber 14 of the syringe barrel 16. When the distal stopper 112 is in the distal-most position, the distal end 20 of the barrel 16 prevents further movement of the distal stopper 112. meaning that continuing to apply pressure to the proximal stopper 110 causes the fluid pressure in the proximal chamber 12 to increase. The increase in fluid pressure causes the distal stopper 112 to transition to the open position, thereby allowing the secondary fluid F2 to be expelled from the syringe 10 through the distal stopper 112 and through the fluid port 22 of the barrel 16.

In a similar manner, the stoppers 110, 112 also move together through the barrel 16 in the proximal direction even though the stoppers 110, 112 are not mechanically connected or engaged together. Specifically, moving the proximal stopper 110 in the proximal direction produces a vacuum or negative pressure in the syringe barrel 16, which draws the distal stopper 112 through the barrel 16 in the proximal direction along with the proximal stopper 110. Accordingly, movement of the distal stopper 112 through the barrel 16 in a proximal direction, as shown by arrow P in FIG. 3B, aspirates the initial fluid F1 into the distal chamber 14 through the fluid port 22 of the barrel 16 in a similar manner as occurs when aspirating a fluid from a vial into a conventional syringe.

The stoppers 110, 112 are configured to move between several positions or configurations during a fluid expulsion procedure. The syringe 10 may initially be provided in a partially-filled configuration, as shown in FIG. 3A, with a secondary fluid F2, such as the second therapeutic agent or flush solution, in the proximal chamber 12. In this initial or partially-filled position, the stoppers 110, 112 are spaced apart from one another by a distance D2 (shown in FIG. 3B) sufficient to contain a volume of the secondary fluid F2 in the proximal chamber 12 of about 1 mL to 20 mL, or preferably about 5 mL to 10 mL. When ready for use, if the distal stopper 112 is not in the distal-most position within the barrel 16, the practitioner can move the distal stopper 112 distally through the barrel 16 to seat the distal stopper 112 against the distal end 20 of the barrel 16. The practitioner can then move the stoppers 110, 112 in the proximal direction (shown by arrow P in FIG. 3B) to aspirate the initial fluid F1 into the distal chamber 14, thereby fully filling the syringe 10, as shown in FIG. 3B.

When the syringe 10 is fully filled, the distal stopper 112 is a distance D1 (shown in FIG. 3B) from the distal end 20 of the barrel 16. Once the syringe 10 is fully filled, moving the stoppers 110, 112 in the distal direction (shown by arrow D in FIG. 3C) moves the distal stopper 112 towards the distal end 20 of the barrel 16, thereby expelling the initial fluid F1 from the distal chamber 14. The syringe 10 is shown in an intermediate position with the full proximal chamber 12 and the empty distal chamber 14 in FIG. 3C. Continued distal movement of the stoppers 110, 112 causes the fluid pressure in the proximal chamber 12 to increase towards the activation or opening pressure for the distal stopper 112. Once the fluid pressure in the proximal chamber 12 exceeds the activation or opening pressure for the distal stopper 112, fluid communication is established between the proximal chamber 12 and the distal chamber 14 through the slit 134 in the body 124 of the distal stopper 112. Once fluid communication between the chambers 12, 14 is established, the secondary fluid F2 is then expelled from the syringe 10 by moving the proximal stopper 110 towards the distal stopper 112. Specifically, movement of the proximal stopper 110 towards the distal stopper 112 causes the secondary fluid F2 to move from the proximal chamber 12 to the distal chamber 14, and then from the distal chamber 14 through the fluid port 22. FIG. 3D shows the syringe 10 in an end-of-use or final position after the fluids F1, F2 are expelled from the syringe barrel 16. In the end-of-use or final position, the distal barrier film 138 adhered to the distal end surface 128 of the distal stopper 112 is in contact with or nearly in contact with the distal end 20 of the barrel 16. Also, the distal end surface 118 of the proximal stopper 110 is in contact with or nearly in contact with the proximal end surface 126 of the distal stopper 112.

With reference again to FIGS. 1A and 1B, as well as the FIGS. 3A-3D, the syringe 10 further comprises a plunger rod 26 connected to the proximal stopper 110 for moving the proximal stopper 110 through the syringe barrel 16. As previously described, moving the proximal stopper 110 through the barrel 16 causes the distal stopper 112 to simultaneously move through the barrel 16 in coordination with the proximal stopper 110, even though the stoppers 110, 112 are not mechanically connected or engaged together. The plunger rod 26 can be, for example, an injection molded part formed from a rigid thermoplastic material, such as polyester, polycarbonate, polypropylene, polyethylene, polyethylene terephthalate, or another thermoplastic material, as are known in the art. The plunger rod 26 can be a conventional plunger rod used in currently available syringes and can be connected to the proximal stopper 110 by standard mechanical connectors, fasteners, or adhesives. In other examples, the plunger rod 26 can be integrally formed or co-molded with the proximal stopper 110.

In some examples, the plunger rod 26 includes a distal end 28 engaged to the proximal stopper 110. For example, as most clearly seen in FIGS. 1B and 3C, the distal end 28 of the plunger rod 26 can include a threaded connector 30 that is inserted into a corresponding cavity 150 (shown in FIGS. 1B and 3C) extending inwardly from a proximal surface of the proximal stopper 110. The plunger rod 26 also includes a proximal end 32 protruding proximally from the proximal end 18 of the syringe barrel 16, and a body 34 extending between the proximal end 32 and the distal end 28 of the plunger rod 26. The proximal end 32 of the plunger rod 26 can include a thumb press plate 36 for manipulating the plunger rod 26 to move the stoppers 110, 112 through the syringe barrel 16. The body 34 of the plunger rod 26 can have a variety of cross-sectional shapes and configurations within the scope of the present disclosure. For example, the body 34 can have a generally cross shaped cross-section. In other examples, the cross-section of the plunger rod 26 can be an I-beam shape, circle, square, L-shaped, or other conventional shapes that can be formed by injection molding processes, as are known in the art. In some examples, the syringe 10 can also include a removable plunger cap 38 (shown in FIG. 3A), such as a partially cylindrical (i.e., a cylindrical body with an axial slot for removing the cap 38 from the syringe 10) or c-shaped spacer, engaged between the thumb press plate 36 of the plunger rod 26 and the proximal end 18 of the barrel 16 for preventing movement of the plunger rod 26 until the removable plunger cap 38 is removed from the syringe 10. The plunger rod cap 38 prevents the plunger rod 26 from being pressed into the syringe barrel 16 at inappropriate times, such as during transport of a prefilled syringe from a manufacturing facility to a customer.

### Method of Expelling Fluid From a Multi-Chamber Syringe

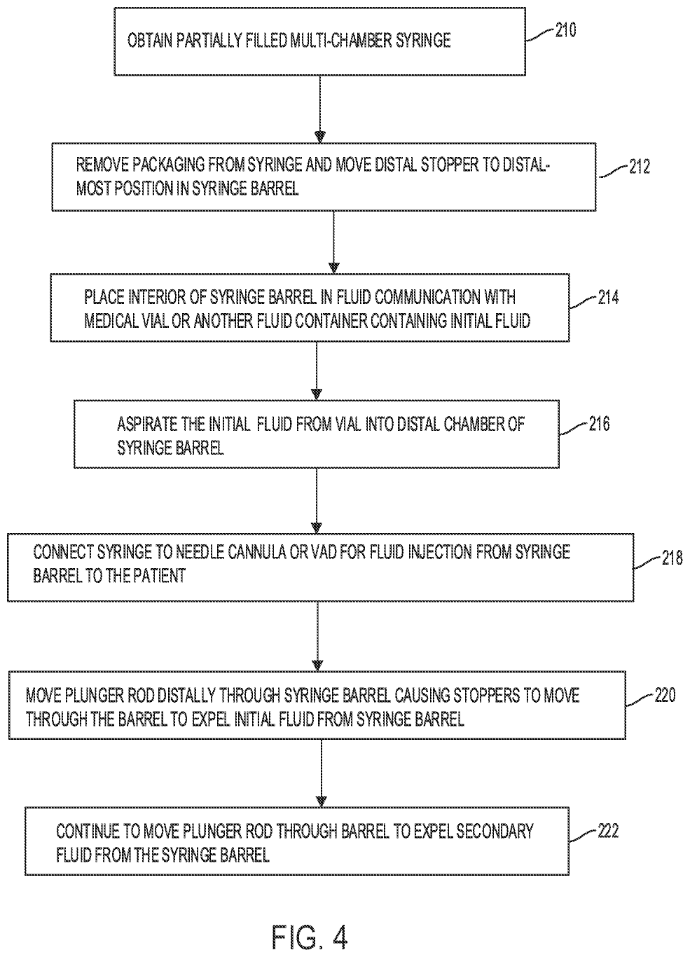

As previously described, the syringe 10 is used for sequential expulsion of fluids, such as expulsion of a medical fluid followed by expulsion of another type of medical fluid or a flush solution, from the syringe 10 to the needle cannula or VAD. A flow chart illustrating steps for sequential expulsion of fluids from the syringe 10 is shown in FIG. 4.

As shown in FIG. 4, at step 210, a practitioner initially obtains a partially filled syringe 10, in which the proximal chamber 12 of the syringe 10 is filled with a predetermined volume of a secondary fluid F2, such as the second type of therapeutic agent or the flush solution. For example, the partially filled syringe 10 can contain about 1 mL to about 20 mL or, preferably, about 5 mL to 10 mL of the therapeutic agent or flush solution. The syringe 10 in the initial or partially-filled configuration is shown in FIG. 3A. At step 212, the practitioner prepares the syringe 10 for use by, for example, removing any packaging from the syringe 10 and removing the plunger rod cap 38 that holds the plunger rod 26 in place. The practitioner may also move the plunger rod 26 in the distal direction to fully seat the distal stopper 112 of the syringe 10 in its distal-most position when the syringe 10 is not initially provided with the distal stopper 112 in the distal-most position. At step 214, the practitioner places the syringe barrel 16 in fluid communication with an interior of a container containing the medical fluid to be delivered to the patient. For example, the practitioner may attach a needle (not shown) to the fluid port 22 at the distal end 20 of the syringe barrel 16 and insert the needle into the container, such as a medical vial, containing the medical fluid to be injected to the patient.

At step 216, the practitioner aspirates the initial fluid F1 (e.g., the medical fluid) into the distal chamber 14 of the barrel 16. For example, the practitioner may grasp the plunger rod 26 and move the plunger rod 26 in the proximal direction, shown by arrow P in FIG. 3B, which moves the stoppers 110, 112 in the proximal direction, thereby aspirating or drawing the initial fluid F1 from an interior of the container into the distal chamber 14 of the syringe barrel 16, as shown by arrows A1 in FIG. 3A. The syringe 10 is shown in its fully filled configuration in FIG. 3B with the initial fluid F1 in the distal chamber 14 and the secondary fluid F2 in the proximal chamber 12.

Once a dose of the initial fluid F1 to be delivered to the patient is drawn into the distal chamber 14, at step 218, the practitioner removes the needle from the fluid port 22 of the syringe barrel 16 and connects the fluid port 22 to another needle cannula with a sharp tip for fluid injection to a patient or to the VAD. For example, the practitioner may insert a nozzle of the syringe barrel 16 into a corresponding port or valve of the VAD, thereby establishing fluid communication between the syringe barrel 16 and a lumen of the VAD. At step 220, once the syringe 10 is appropriately connected to the needle cannula or VAD, the practitioner grasps the plunger rod 26 and pushes the plunger rod 26 in the distal direction (shown by arrow D in FIG. 3B), which causes the stoppers 110, 112 to move distally through the syringe barrel 16. Distal movement of the distal stopper 112 causes the initial fluid F1 (e.g., the medical fluid) in the distal chamber 14 of the syringe barrel 16 to be expelled from the syringe barrel 16, as shown by arrow A2 in FIG. 3B, to the needle cannula or VAD through the fluid port 22 or nozzle of the syringe barrel 16. The practitioner continues to move the plunger rod 26 in the distal direction until the distal stopper 112 is seated in the syringe barrel 16 at its distal-most position. The distal stopper 112 is shown in this distal-most position, with the stopper 112 in the closed position, in FIG. 3C.

In order to cause the distal stopper 112 to transition to the open position and to establish fluid communication between the proximal chamber 12 and the distal chamber 14, the practitioner continues to move the plunger rod 26 in the distal direction. As previously described, the force required to cause the stopper 112 to open may be greater than the force required to move the stoppers 110, 112 through the barrel 16. Accordingly, once the distal stopper 112 is in the distal-most position, the practitioner may need to apply additional force to the plunger rod 26 to open the distal stopper 112. Further, as previously described, the distal movement of the plunger rod 26 can be a single continuous stroke in the distal direction performed using one hand (e.g., single-handed operation). As such, the practitioner does not need to perform any other action other than continuing distal movement of the plunger rod 26 to automatically open the distal stopper 112. More specifically, with the distal stopper 112 in the distal-most position, the continued distal movement of the plunger rod 26 causes the proximal stopper 110 to move distally through the barrel 16 towards the distal stopper 112. This distal movement of the proximal stopper 110 increases the fluid pressure in the proximal chamber 12 towards the activation or opening pressure of the distal stopper 112. Once the fluid pressure in the proximal chamber 12 reaches or exceeds the activation or opening pressure of the distal stopper 112, the distal stopper 112 opens to establish fluid communication between the proximal chamber 12 and the distal chamber 14 through the distal stopper.

At step 222, once fluid communication between the proximal chamber 12 and the distal chamber 14 is established, the practitioner continues to push the plunger rod 26 in the distal direction, which moves the proximal stopper 110 through the syringe barrel 16 in the distal direction towards the proximal surface 126 of the distal stopper 112. As shown by arrows A3 (in FIG. 3C), movement of the proximal stopper 110 towards the distal stopper 112 causes the secondary fluid F2, such as the second therapeutic agent or flush solution, in the proximal chamber 12 to pass through the distal stopper 112 and into the distal chamber 14. The secondary fluid F2 then passes through the distal chamber 14 and is expelled from the syringe barrel 16 to the needle cannula or VAD through the fluid port 22 at the distal end 20 of the syringe barrel 16. The syringe 10 is shown in an end-of-use or final position in FIG. 3D, with the distal end surface 118 of the proximal stopper 110 in contact with or nearly contacting the proximal end surface 126 of the distal stopper 112.

### Molding Methods for Stoppers for Multi-Chamber Syringes

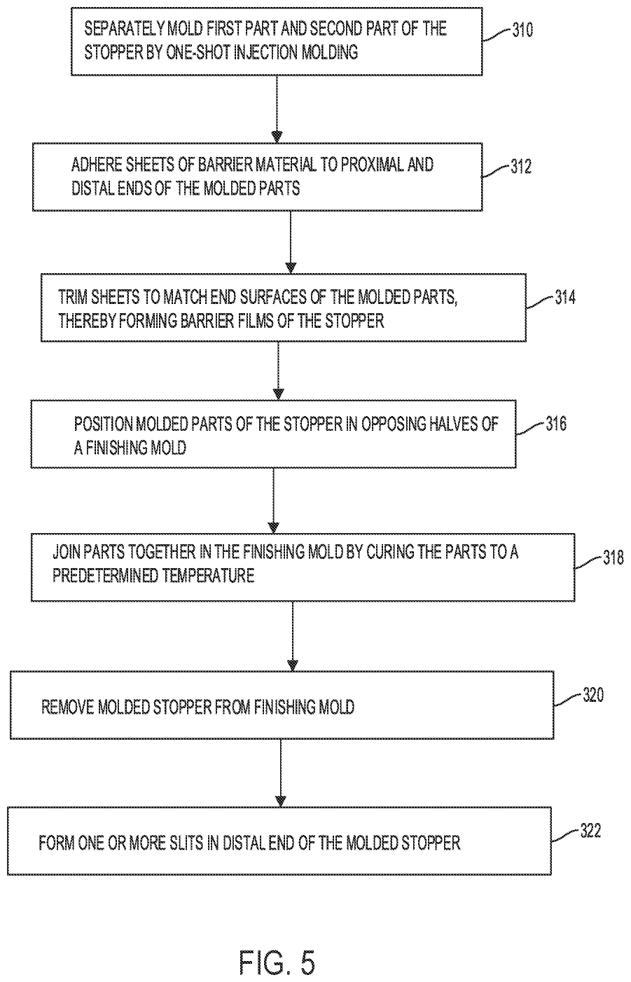

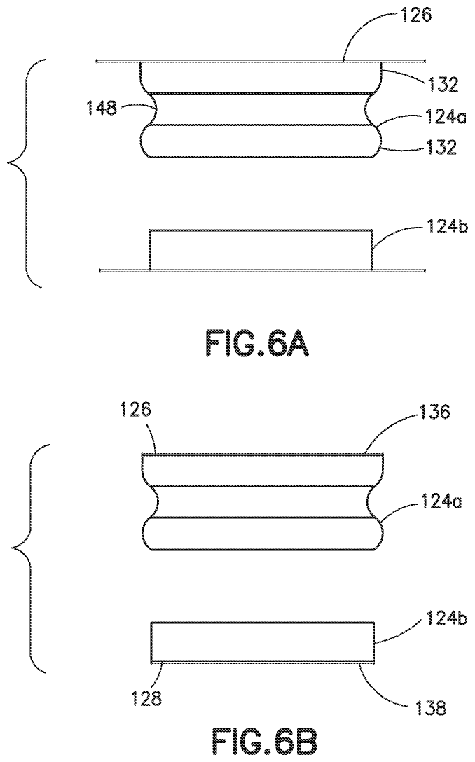

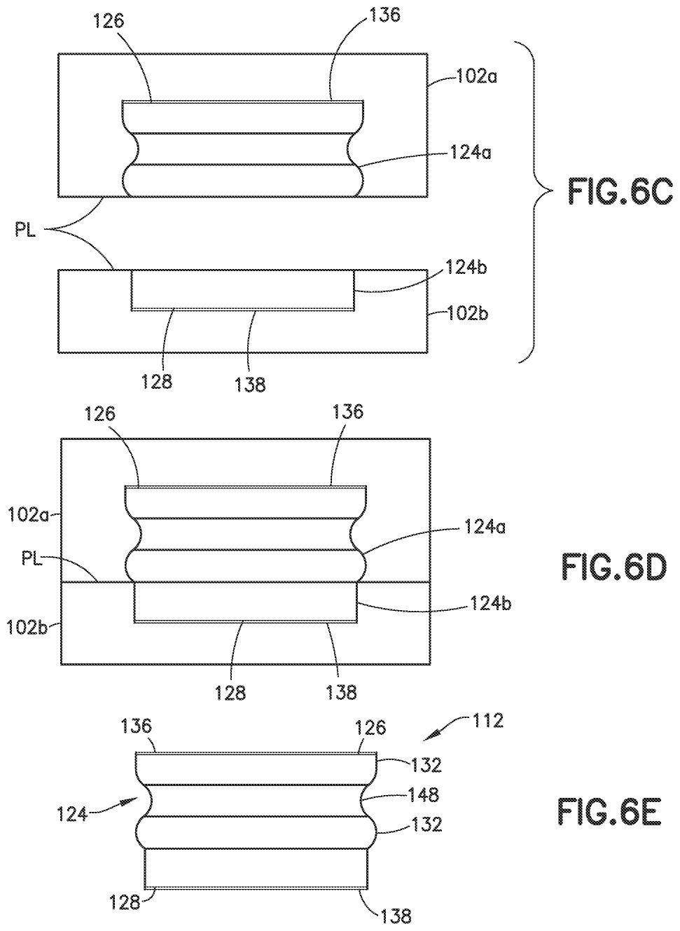

The distal stopper 112 for the multi-chamber syringe 10 of the present disclosure can be made by a modified one-shot injection molding method that produces a molded stopper comprising the integral body 124 formed from two or more parts or sections. As previously described, the stopper 112 also includes the proximal barrier film 136 adhered or laminated to the proximal end surface 126 of the integral body 124 and the distal barrier film 138 adhered or laminated to distal end surface 128 of the integral body 124. FIG. 5 is a flow chart showing steps of the molding method for making the distal stopper 112. FIGS. 6A-6E are schematic drawings showing the steps of the molding method.

At step 310, the molding method initially comprises molding a first or proximal part 124a and a second or distal part 124b, which will be joined together to form the body 124 of the distal stopper 112. Schematic drawings of the parts 124a, 124b are shown in FIGS. 6A-6D. The parts 124a. 124b can be formed by a one-shot injection molding process in separate molds from a precursor material, which can be cured to form a thermoplastic elastomeric part. As previously described, the thermoplastic elastomer can comprise silicone, polypropylene, polyethylene, or synthetic or natural rubber (e.g., isoprene), or combinations thereof. In some examples, the first or proximal part 124a includes two annular ribs 132 with the recessed portion 148 between the ribs 132. The distal part 124b can be narrower having a peripheral surface that is not wide enough to contact the inner surface of the syringe barrel 16. For example, a diameter of the distal part 124b can be about the same as a diameter of the recessed portion 148 of the proximal part 124a. The parts 124a, 124b can be partially cured, such that the parts 124a, 124b are solid enough to be removed from molds, but are sufficiently tacky to be joined together in a subsequent final molding step. In step 310, each of the parts are cured to the rheological point. In step 318, this cure cycle is replicated.

At step 312, after the parts 124a. 124b are molded and partially cured, the method further comprises adhering sheets of barrier material to the parts 124a, 124b to form the proximal barrier film 136 and the distal barrier film 138. Specifically, as shown in FIG. 6A, a sheet of the barrier material is applied over the proximal end surface 126 on the first part 124a and a second sheet of the barrier material is applied over the distal end surface 128 of the second part. For example, the barrier sheets can be adhered to the parts 124a, 124b by tackiness of the partially cured parts 124a, 124b. The sheets may also be adhered to the parts 124a, 124b using a biocompatible adhesive or an amount of uncured polymer material applied to the surfaces 126, 128 of the parts 124a, 124.

At step 314, the method further comprises trimming the barrier sheets to form the barrier films 136, 138. For example, as shown in FIG. 6B, the sheet over the proximal end surface 126 of the proximal part 124a can be trimmed to a diameter matching the proximal end surface 126 to form the proximal barrier film 136. The sheet covering the distal end surface 128 of the distal part 124b can be trimmed to a diameter matching the distal end surface 128 of the distal part 124b to form the distal barrier film 138.

At step 316, the parts 124a, 124b and barrier films 136, 138 adhered thereto are then positioned in opposing halves or parts 102a, 102b of a finishing mold, as shown in FIG. 6C. Specifically, the parts 124a, 124b are positioned with the barrier films 136, 138 in contact with surfaces of the mold parts and with an uncured surface of the parts 124a. 124 positioned in an interior of the mold. At step 318, as shown in FIG. 6D, the mold parts 102a, 102b are brought together such that the uncured surfaces of the parts 124a, 124b are in contact with each other along a parting line PL (shown in FIGS. 6C and 6D). In some examples, the parting line of the mold can be relieved slightly, such as by about 0.05 mm to about 0.2 mm. The mold is then cured to a temperature of about 50° C. to about 70° C. in order to fully cure the parts 124a, 124b, thereby forming an integral body 124 from the parts 124a, 124b. Optionally, the pressure applied to the mold to fully cure the parts is on the order of from 30-250 bars, depending on size and shape. Typically, butyl blends may take from around 2-8 minutes in total, and may be divided between first and final molding steps.

At step 320, after curing, the molded part is removed from the finishing mold, as shown in FIG. 6E. Specifically, the molded part is a stopper 112 that includes the integrally molded body 124 with the proximal barrier film 136 adhered or laminated to the proximal end surface 126 of the body 124 and the distal barrier film 138 adhered or laminated to the distal end surface 128. At step 322, the method further comprises forming the slit 134 in the distal end surface 128 of the body 124, thereby forming the stopper 112 shown, for example, in FIGS. 2A and 2B.

While examples of the stoppers, multi-chamber syringes, and methods of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A stopper for a multi-chamber syringe, the stopper comprising:
a body comprising a first end surface, a second end surface, a peripheral surface extending between the first end surface and the second end surface, and at least one slit extending through the body for permitting fluid flow through the body of the stopper;
a first barrier film adhered to the first end surface of the body; and
a second barrier film adhered to the second end surface of the body,
wherein the first barrier film and/or the second barrier film comprise slits or openings aligned with the slit extending through the body for permitting fluid flow through the stopper.

2. The stopper of claim 1, further comprising at least one protrusion extending axially from the proximal end of the body for separating the stopper from another stopper of a multi-chamber syringe.

3. The stopper of claim 1, wherein the peripheral surface of the body comprises at least one rib configured to contact an inner surface of a sidewall of a syringe barrel and at least one recessed portion configured to be spaced apart from the inner surface of the sidewall of the syringe barrel.

4. The stopper of claim 1, wherein the first stopper and/or the second stopper comprise a thermoplastic elastomer comprising at least one of silicone, polypropylene, polyethylene, synthetic rubber, natural rubber, or combinations thereof.

5. The stopper of claim 1, wherein the body further comprises a cavity extending axially through a portion of the body from the first end surface toward the second end surface of the body.

6. The stopper of claim 5, wherein the cavity comprises an open first end, a closed second end with a substantially flat second surface, and an annular side surface between the first end and the second end, wherein the annular side surface comprises concave and convex portions.

7. The stopper of claim 6, wherein the slit extending through the body extends between the flat second surface of the cavity and the second end surface of the body.

8. The stopper of claim 1, wherein the body is made from two separately molded parts mated together by a subsequent molding process.

9. The stopper of claim 1, wherein the stopper is configured to transition between (i) a closed position, where fluid flow through the slit of the body is prevented, when a fluid pressure in the syringe barrel is below a predetermined opening pressure, and (ii) an open position when the fluid pressure in the syringe barrel is greater than or equal to the predetermined opening pressure, thereby establishing fluid communication between chambers of a syringe barrel through the stopper.

10. The stopper of claim 1, wherein the slit of the body is biased to and initially provided in the closed position.

11. The stopper of claim 1, wherein the first barrier film and/or the second barrier film comprise disks with a diameter matching a diameter of the first end surface or the second end surface of the body.

12. The stopper of claim 1, wherein the first barrier film extends over an annular surface of the body and surfaces of a cavity defined by the body.

13. The stopper of claim 1, wherein the first barrier film and/or the second barrier film comprise a fluoropolymer.

14. The stopper of claim 1, wherein the peripheral surface of the body is free from barrier films.

15. The stopper of claim 1, wherein the peripheral surface of the body is uncoated.

16. The stopper of claim 1, wherein the first barrier film and/or the second barrier film have a thickness of from about 0.013 mm to about 0.25 mm.

17. A multi-chamber syringe for sequential expulsion of at least an initial fluid followed by a secondary fluid, the syringe comprising:
a barrel comprising a first end, a second end comprising a fluid port for expulsion of the initial fluid and the secondary fluid from the barrel, and a sidewall extending between the first end and the second end of the barrel;
a first stopper slidably positioned in the barrel; and
a second stopper comprising the stopper of claim 1 slidably positioned in the barrel,
wherein the first stopper and the second stopper define a first chamber of the syringe barrel between the first stopper and the second stopper configured to contain the secondary fluid and a second chamber of the syringe barrel between the second stopper and the second end of the syringe barrel configured to contain the initial fluid.

18. A molding method for the stopper of claim 1, the method comprising:
molding a first part and a second part of the body of the stopper to a partially molded state;
connecting a first barrier film to a first end of the partially-molded first part;
connecting a second barrier film to a second end of the partially-molded second part;
adhering the first part to the second part in a finishing mold, thereby forming the stopper comprising the body with the first and second barrier films laminated to the body; and
forming the slit through the body and through the first barrier film and/or the second barrier film.

19. The method of claim 18, wherein the first part and the second part are molded by a one-shot injection molding process.

20. The method of claim 18, wherein connecting the first barrier film to the first molded part comprises attaching a flat sheet of a barrier material to the first end of the first part and trimming the flat sheet to match a shape of the first end of the first part, and
wherein connecting the second barrier film to the second end of the second part comprises attaching a flat sheet of a barrier material to the second end of the second part and trimming the flat sheet to match a shape of the second end of the second part.
